(19) United States
(12) Patent Application Publication
(10) Pub. No.: US 2024/0016392 A1
(43) Pub. Date: Jan. 18, 2024

(54) METHOD AND SYSTEM FOR SCREENING A USER'S TEMPERATURE

(71) Applicant: SITA Advanced Travel Solutions Limited, Hampshire (GB)

(72) Inventors: Christian HALET, Brussels (BE); Michael DOUGHTY, The Hague (NL); Kevin O'SULLIVAN, Hampshire (GB)

(21) Appl. No.: 18/357,765

(22) Filed: Jul. 24, 2023

Related U.S. Application Data
(63) Continuation of application No. PCT/EP2022/052140, filed on Jan. 28, 2022.

(30) Foreign Application Priority Data
Jan. 28, 2021 (GB) 2101183.8

(51) Int. Cl. A61B 5/01; G06V 40/16; H04N 23/11

(52) U.S. Cl. CPC A61B 5/015; G06V 40/166; H04N 23/11

(57) ABSTRACT

A method for screening a user temperature, the method comprising: detecting a user's face in a first image using a first camera operating within a first portion of the electromagnetic spectrum; aligning the detected user's face to a second image of the user's face detected using a second camera operating within a second portion of the electromagnetic spectrum which is different from the visible spectrum; determining the user's temperature based on the second image aligned to the first image; and determining if the user's temperature exceeds a predetermined threshold.

### CROSS-REFERENCE TO RELATED APPLICATIONS

This application is a continuation of International Patent Application No. PCT/EP2022/052140, filed on Jan. 28, 2022 and entitled “A METHOD AND SYSTEM FOR SCREENING A USER'S TEMPERATURE,” which claims the benefit of and priority of: GB Patent Application No. 2101183.8, filed Jan. 28, 2021 and entitled “A METHOD AND SYSTEM FOR SCREENING A USER'S TEMPERATURE,” which are each incorporated by reference herein in their entireties.

### FIELD OF THE INVENTION

The present invention relates to a method and system for screening a user's temperature.

### BACKGROUND OF THE INVENTION

The identification of users with an elevated skin temperature (EST) can be used to determine if a person should be subjected to health screening checks. EST may be indicative of a health condition, such as COVID-19. Identification of ESTs may be particularly applicable at a transportation hub such as an airport or a train station.

Existing infrared cameras are able to identify human bodies and identify their skin temperature in a standalone setup. Such technology has been integrated into devices such as kiosks which may be located at a transportation hub. Device providers have integrated this technology into kiosks and access gates located in, for example, an airport, and are designed to prevent a user proceeding further if is detected that they have an EST.

Typically, existing devices require control of an operator when in use. In addition, the use of such devices typically relies on funnels to densify users (such as passengers in a transportation hub) together to get any kind of significant throughput. For various reasons including safety and convenience, increasing density of passengers in a given space may be undesirable.

Temperature identification technology has been implemented on a limited scale into self-service devices and kiosks, but lacks the scalability to be implemented more widely.

### SUMMARY OF THE INVENTION

Inventors of the present invention have appreciated that existing technology to detect EST may be improved upon and indeed scaled to be implemented more widely.

The invention is defined by the independent claims to which reference should now be made. Optional features are set forth in the dependent claims.

Arrangements of the present disclosure provide a temperature screening system integrated with various touchpoints, for example at an airport, with remote user notification via an electronic device, and having cross device confirmation by checking an approval credential such as temperature exceeding a predetermined threshold.

Arrangements of the present disclosure combine the integration of temperature screening technology into a plurality of touchpoints across a transportation hub, which may non-exhaustively include: terminal or area entry Facepods; check-in kiosks; bag tagging kiosks; automated bag drops; restricted access gates; lounge entry Facepods; automated assisted boarding gates; and self-service and non-self-service kiosks.

According to an aspect of the present invention, there is provided a method for screening a user temperature, the method comprising: detecting a user's face in a first image using a first camera operating within a first portion of the electromagnetic spectrum; aligning the detected user's face to a second image of the user's face detected using a second camera operating within a second portion of the electromagnetic spectrum which is different from the first portion of the electromagnetic spectrum; determining the user's temperature based on the second image aligned to the first image; and determining if the user's temperature exceeds a predetermined threshold. A user's temperature may be measured and compared against a predetermined threshold to establish whether a user has an elevated skin temperature. If the user's temperature is above the predetermined threshold, it may be considered that the user has an EST. If the user's temperature is below the predetermined threshold, it may be considered that the user does not have an EST. Where the user temperature equals the predetermined threshold it may be considered that the user temperature is an EST or not an EST. The threshold may be determined by averaging temperatures measured on a set of users or passengers, and then adding an offset to calculate a threshold. This calibration step advantageously suppresses differences between different cameras.

Preferably, the second image is associated with thermal image data. Further preferably the first image is associated with the visible spectrum image. More preferably, the user's face in the thermal image is located using a thermal face location algorithm. In particular, a face location algorithm may use the infra-red stream, rather than the visible stream, to determine the location of the face within the second image. According to embodiments of the invention this may improve accuracy. An exemplary algorithm may be found here: https://github.com/maxbbraun/thermal-face. Embodiments of the invention may use TFLite Object Detection.

The first portion of the electromagnetic spectrum may be the visible spectrum, and the second portion of the electromagnetic spectrum may be the thermal spectrum. The visible spectrum may comprise a wavelength range of around 380 to 750 nanometers. The thermal spectrum may comprise a wavelength range of around 1,000 to 14,000 nanometers. In the following description, the first and second cameras will be referred to as operating within the visible and thermal spectrums respectively, but it will be appreciated that they are not limited as such.

The method may be implemented at a transportation hub. For example, the method may be implemented at one or more touchpoints at a transportation hub. A touchpoint may be any location at a transportation hub which requires a user to come into contact with a device, checkpoint, or any location such as those listed non-exhaustively above. By implementing this method, a user's temperature is checked and it can be determined whether the user's temperature is considered elevated and therefore that user should not proceed through the transportation hub until they have been subject to further health screening. Preferably, the method is implemented at a plurality of locations at a transportation hub and the temperature of a plurality of users, such as passengers, is taken. By establishing if the temperature of each of a plurality of passengers, and preferably all of the passengers in a transportation hub, is elevated or not, passengers with elevated temperature can be prevented from proceeding in order to improve safety at the transportation hub. The passenger may only be able to proceed once their health has been confirmed.

The aligning the detected user's face to the second image of the user's face detected using the second camera may comprise using rotational information from the first camera, the rotational information being indicative of rotation of the user's face about an axis. The aligning may comprise aligning and/or mapping. The aligning the detected user's face using the second camera operating within the thermal spectrum may comprise locating the user's face in the second image using a face location algorithm. The face location algorithm may be an algorithm such as Thermal Face.

The first camera may have a larger horizontal and/or vertical field of view than the second camera. An alignment algorithm configured to align the user's face between the two images may be calculated by taking a plurality of samples where a user's face is located at different positions and distances. A linear regression may be calculated for both X and Y coordinates corresponding to facial features between images taken on the first and second cameras. When considering the Y coordinate, an influence of eye distance may be accounted for. For the X coordinate, a mapping from a thermal camera to a visible camera may be of the form: Xthermal=AXvisible+B, and the Y coordinate mapping may be of the form: Ythermal=AYvisible+B(EyeDistancevisible)+C, where A, B, and C are constants to be calculated for a first and second camera, and account for non-alignment between the two cameras. Any appropriate software may be implemented to apply the alignment algorithm to accurately map between the two images. As described above, rotational information from the first camera is provided to account for any rotation of the user's face.

Effectively aligning the first image and the second image is advantageous as it allows accurate location of the user's face and therefore effective measurement of the user's temperature. When imaging the user's face, there is a parallax effect present dependent on the distance between the user and the camera. Identifying face location on the thermal image advantageously allows this parallax effect to be minimized or ignored entirely. Typically, algorithms employed to locate facial features on an image taken with a thermal camera are inaccurate as facial features are often difficult to locate. This is due to glasses often being fully reflective of infrared wavelengths, and also due to low contrast (due to a low temperature difference) over the thermal image. In addition, the eye location is typically very dark or black in a thermal image. The inventors of the present invention have appreciated the difficulty in aligning images from the two cameras but arrangements are provided in which this is possible.

The second camera, operating within the thermal spectrum, or thermal camera, may capture an image of the user's face and locate the position of the user's face using a face location algorithm. Temperature of the user may be measured over a forehead area. Therefore, if the user's head is rotated about an axis, the forehead will be non-central and require alignment with the thermal image. Rotational information from the first camera may be used for this alignment. Rotational information from the first camera may comprise the degree of rotation about an axis of a user's face. The axis may be any appropriate axis, such as a horizontal axis, defining an origin point at which a user's face is normally positioned. The rotational information may then be used to align the user's face located in the thermal image with the user's face in the first image. The aligning the detected user's face to the second image of the user's face detected using the second camera may comprise aligning a portion of the user's face to the second image. The portion may be a forehead portion. This allows accurate location of the user's forehead at which temperature can be taken. This advantageously avoids temperature being taken of any other portion of the user's face. For example, if there is rotation of the user's face but the thermal camera does not account for this rotation and thereby assumed the forehead is at a central, standard position, temperature may be taken of a portion of the user's face that is not the forehead, for example over an eye. This would provide an unwanted and incorrect temperature measurement. This is very undesirable given the health and safety implementations of the arrangements of the present disclosure.

The user's temperature may be calculated by taking a region, for example the forehead region, and excluding one or more pixels such as two pixels with the highest temperature. The temperature of one or more pixels such as five pixels with the highest temperature of the non-excluded pixels may then be averaged. Temperature may be calculated over a plurality of frames, for example five frames. An offset may be applied to compensate for skin temperature (such as at the forehead) deviation compared to internal body temperature. The offset may be calibrated with one or more user's, and may be around 3 degrees Celsius.

The method may further comprise generating a user temperature output indicative of whether the user's temperature exceeds the predetermined threshold. In other words, the method may further comprise returning a binary result based on the pre-programmed threshold, the binary result comprising a positive or negative output indicating whether the user has an EST or not, respectively. The method may further comprise outputting the user temperature output indicative of whether the user's temperature exceeds the predetermined threshold to a remote user on an electronic device. A remote user may use the electronic device to monitor one or more locations at which a user temperature is being determined. In this way, the remote user may be notified of any determined ESTs. The operator may then approach a user and subject them to further health screening. Based on the user temperature output, the remote user may: allow the user to proceed further if the user's temperature does not exceed the predetermined threshold; or not allow the user to proceed further if the user's temperature exceeds the predetermined threshold. Proceed further may mean anything that allows a user to continue their path or journey through a location such as a transportation hub. For example, it may mean to proceed past a given checkpoint, such as proceed through an entry point in a terminal, or may mean allowing the user to check in baggage at a transportation hub, or receive tickets or boarding passes. The operator may also reset touchpoint or device at which the user was attempting to proceed past, such as an entry point.

The method may also be implemented at a self-service device or kiosk. This advantageously removes or at least limits the requirement for operator or remote user input and allows the maintenance of low user or passenger density. Operator assistance may be via remote mobile notifications not requiring flow or access control. The method may further comprise the self-service device: allowing the user to proceed further if the user's temperature does not exceed the predetermined threshold; or not allowing the user to proceed further if the user's temperature exceeds the predetermined threshold. For example, the self-service device may be an area entry Facepod. The Facepod may be configured to run a verification application. If the user does not have an EST, the user may be granted entry. If the user does have an EST, the user may not be granted entry. By applying this in a plurality of self-service devices, the infrastructure is scaled to establish temperatures of a plurality of users without requiring a densification of users to, for example, a single check in desk. An operator may oversee the self-service device or a plurality of self-service devices. The operator may be notified of an EST and at which touchpoint or self-service device the EST was detected through the electronic device. The operator may then approach a user and assist them with further health screening checks. The operator may also manually allow the user to proceed if the operator is notified that the user does not have an EST, for example through an entry gate. The operator may also reset the touchpoint or self-service device.

The method may further comprise storing the user's temperature in a database. The user temperature may be stored together with corresponding user information. That is, the user temperature may be stored with information about the user to which the temperature relates such as personal data or biometrics which may include an image of the user. This advantageously allows for touchpoints to be located outside of a transportation hub such as a terminal building, thereby reducing the risk of passenger crowding and queueing. This allows passengers who would not visit another touchpoint (such as a check-in kiosk or automated bag drop) to proceed directly through the process and utilise touchpoints which may not have temperature detection technology integrated.

The method may further comprise: accessing the database; retrieving the stored user temperature; and allowing the user to proceed further if the user's temperature does not exceed the predetermined threshold, or not allowing the user to proceed further if the user's temperature does exceed the predetermined threshold. A remote user may access the database using a computing device or electronic device. For example, a user may use a touchpoint which does not have temperature technology integrated. In order to confirm that the user does not have an EST, a remote user may access the database and retrieve a temperature of the user previously taken at another touchpoint, for example outside of the location or transportation hub. This may be done by interrogating the database with personal details and/or performing a biometric search. This provides a convenient experience for the user as their temperature need not be taken again. Conversely, it also means a passenger with an EST may be stopped at a touchpoint which does not have the technology to take their temperature. The database may also be accessed by one or more operators using electronic devices or computing devices which are portable. Access to the database may be provided to a transportation hub operator such as an airport operator, and airlines and government agencies to ensure compliance with local and destination laws and regulations.

Arrangements of the present invention provide flexibility, accounting for changing regulations and/or individual customer needs at the same location, through the use of an infrastructure of self-service and non-self-service touchpoints equipped with the temperature detection technology, as well as a database storing determined user temperatures. This allows for safe and convenient travel for users through a location such as a transportation hub.

According to another aspect of the disclosure, there is provided a method for screening a user temperature, the method comprising: detecting a user's face in a first image using a first camera operating within a first portion of the electromagnetic spectrum; aligning the detected user's face to a second image of the user's face detected using a second camera operating within a second portion of the electromagnetic spectrum which is different from the first portion of the electromagnetic spectrum; and determining the user's temperature based on the second image aligned to the first image.

According to another aspect of the disclosure there is provided a computer program for performing any of the methods as described above.

According to another aspect of the disclosure there is provided a computer program product comprising instructions which, when the program is executed by a computer, cause the computer to carry out any of the methods as described above.

According to another aspect of the present invention there is provided a system for screening a user temperature, the system comprising: a first camera operating within a first portion of the electromagnetic spectrum and being configured to detect a user's face in a first image; a second camera operating within a second portion of the electromagnetic spectrum which is different from the first portion of the electromagnetic spectrum, the second camera being configured to detect the user's face in a second image; an aligner configured to align the user's face detected in the first image to the user's face detected in the second image; and one or more determiners configured to determine: the user's temperature based on the second image aligned to the first image; and if the determined user's temperature exceeds a predetermined threshold. The system may be implemented at one or more touchpoints throughout a location such as a transportation hub. The one or more determiners may be applied at the touchpoint, or separately from the touch point, or both. A single determiner may determine the user's temperature and if the temperature exceeds the predetermined threshold, or these may be determined by separate determiners. The first and second cameras may be integrated into the touchpoint. The touchpoint may be any touchpoint, for example the touchpoint may non-exhaustively include: terminal or area entry Facepods; check-in kiosks; bag tagging kiosks; automated bag drops; restricted access gates; lounge entry Facepods; automated assisted boarding gates; and self-service and non-self-service kiosks. The system may be integrated into the touchpoint to perform elevated skin temperature detection on a user such as a passenger before they finalize their transaction or attempt to proceed past the touchpoint. The system may be implemented at a plurality of touchpoints at a location. This enables user temperatures to be determined at various touchpoints, removing the need for densifying passengers.

The aligner may be further configured to align the user's face detected in the first image to the user's face detected in the second image using rotational information from the first camera, the rotational information being indicative of rotation of the user's face about an axis. The second camera may be configured to detect the user's face in a second image using a face location algorithm. Effectively aligning the first image and the second image is advantageous as it allows effective measurement of the user's temperature.

The second camera, operating within the thermal spectrum, or thermal camera, may be configured to capture an image of the user's face and locate the position of the users face using a face location algorithm. Temperature of the user may be measured over a forehead area. Therefore, if the user's head is rotated about an axis, the forehead of the user will be non-central and require alignment with the thermal image. Rotational information from the first camera may be configured to be used for this alignment. Rotational information from the first camera may comprise the degree of rotation about an axis of a user's face. The rotational information may then be configured to be used to align the user's face located in the thermal image with the user's face in the first image. The aligner may be configured to configured to align a portion of the user's face detected in the first image to the user's face detected in the second image. The portion may be a forehead portion.

The system may further comprise an output configured to output a user temperature output indicative of whether the user's temperature exceeds the predetermined threshold to a remote user on an electronic device. The output may be integrated to the touchpoint or may be separate. The output may be connected to a network through a wired and/or wireless connection. The wireless connection may be over a wireless local area network (WLAN) such as a Wi-Fi network, or may be over Bluetooth. This allows the output to be sent to any other electronic or computer device connected to the network. The electronic device or computer device used by the remote user may be any suitable electronic device, for example a mobile telephone device, tablet device, or laptop of personal computer device.

The electronic device may be configured to receive an input from the remote user: allowing the user to proceed further if the user's temperature does not exceed the predetermined threshold; or not allowing the user to proceed further if the user's temperature exceeds the predetermined threshold. In other words, the remote user may be notified that a user temperature exceeds the predetermined threshold and therefore is considered an EST. The remote user may then input into the electronic device a control input preventing the user from proceeding, for example through an entry gate. Alternatively, if the user's temperature is below the predetermined threshold and is not considered elevated, the remote user may provide an input allowing the user to proceed, for example through the entry gate. This may be particularly applicable when the system is integrated into a non-self-service device or kiosk.

The system may be integrated into a self-service device or kiosk. The self-service device or kiosk may be configured to: allow the user to proceed further if the user's temperature does not exceed the predetermined threshold; or not allow the user to proceed further if the user's temperature exceeds the predetermined threshold. Similarly to above, where it is determined that the user's temperature is above the predetermined threshold and therefore is an EST, the self-service device may prevent the user from proceeding. For example the self-service kiosk may prevent the user from passing through an entry gate, prevent the user from checking in their bags, or prevent the user from receiving tickets or boarding passes. Conversely, where it is determined that the user does not have an EST and their temperature is below the predetermined threshold, the user may be allowed to proceed, such as being granted access through a gate, being allowed to check in bags, or being allowed to receive tickets or boarding passes.

The system may further comprise a database configured to store the user's temperature. The database may be configured to store user information corresponding to the user's temperature. That is, the user temperature may be stored with information about the user to which the temperature relates such as personal data or biometrics which may include an image of the user. The system may be integrated into one or more touchpoints outside of the location or transportation hub, and the provision of the database advantageously allows for these touchpoints to be located outside of a transportation hub such as a terminal building. This thereby reduces the risk of passenger crowding and queueing. It allows passengers who would not visit another touchpoint (such as a check-in kiosk or automated bag drop) to proceed directly through the process and utilise touchpoints which may not have temperature detection technology integrated.

The system may further comprise one or more computing devices configured to: access the database; retrieve the stored user temperature; and allow the user to proceed further if the user's temperature does not exceed the predetermined threshold, or not allow the user to proceed further if the user's temperature does exceed the predetermined threshold. Each of the one or more computing devices may each perform each step or accessing the database, retrieving the stored user temperature, and allowing or not allowing the user to proceed, for example for different users, or each of the one or more computing devices may only perform a subset of the steps such as one step. The one or more computing devices may be any computing device or electronic device capable of accessing the database and may be connected to the network of the location or transportation hub. The one or more computing devices may comprise the electronic device used by the remote user. The one or more computing devices may comprise, for example, a personal computer device or laptop device located at a check in desk which, for example, may lack the temperature detection technology. A remote user may access the database using the one or more computing devices or electronic devices. In order to confirm that the user does not have an EST, a remote user may access the database and retrieve a temperature of the user previously taken at another touchpoint, for example outside of the location or transportation hub. This may be done by interrogating the database with personal details and/or performing a biometric search. The biometric search comprise retrieving a measured temperature, or associating a biometric with an existing record. This provides a convenient experience for the user as their temperature need not be taken again. Conversely, it also means a passenger with an EST may be stopped at a touchpoint which does not have the technology to take their temperature. The database may also be accessed by one or more operators using electronic devices or computing devices which are portable. Access to the database may be provided to a transportation hub operator such as an airport operator, and airlines and government agencies to ensure compliance with local and destination laws and regulations.

According to another aspect of the disclosure there is provided a system for screening a user temperature, the system comprising: a first camera operating within a first portion of the electromagnetic spectrum and being configured to detect a user's face in a first image; a second camera operating within a second portion of the electromagnetic spectrum which is different from the first portion of the electromagnetic spectrum, the second camera being configured to detect the user's face in a second image; and an aligner configured to align the user's face detected in the first image to the user's face detected in the second image.

From the foregoing, it will be appreciated that the system may comprise a computer processor running one or more server processes for communicating with client devices. The server processes comprise computer readable program instructions for carrying out the operations of the present invention. The computer readable program instructions may be or source code or object code written in or in any combination of suitable programming languages including procedural programming languages such as C, object orientated programming languages such as C #, C++, Java, scripting languages, assembly languages, machine code instructions, instruction-set-architecture (ISA) instructions, and state-setting data.

The wired or wireless communication networks described above may be public, private, wired or wireless network. The communications network may include one or more of a local area network (LAN), a wide area network (WAN), the Internet, a mobile telephony communication system, or a satellite communication system. The communications network may comprise any suitable infrastructure, including copper cables, optical cables or fibres, routers, firewalls, switches, gateway computers and edge servers.

The system described above may comprise a Graphical User Interface. Embodiments of the invention may include an on-screen graphical user interface. The user interface may be provided, for example, in the form of a widget embedded in a web site, as an application for a device, or on a dedicated landing web page. Computer readable program instructions for implementing the graphical user interface may be downloaded to the client device from a computer readable storage medium via a network, for example, the Internet, a local area network (LAN), a wide area network (WAN) and/or a wireless network. The instructions may be stored in a computer readable storage medium within the client device.

As will be appreciated by one of skill in the art, the invention described herein may be embodied in whole or in part as a method, a system, or a computer program product including computer readable instructions. Accordingly, the invention may take the form of an entirely hardware embodiment or an embodiment combining software, hardware and any other suitable approach or apparatus.

The computer readable program instructions may be stored on a non-transitory, tangible computer readable medium. The computer readable storage medium may include one or more of an electronic storage device, a magnetic storage device, an optical storage device, an electromagnetic storage device, a semiconductor storage device, a portable an erasable programmable read-only memory (EPROM or Flash memory), a static random access memory (SRAM), a portable compact disc read-only memory (CD-ROM), a digital versatile disk (DVD), a memory stick, a floppy disk.

Exemplary embodiments of the invention may be implemented as a circuit board which may include a CPU, a bus, RAM, flash memory, one or more ports for operation of connected I/O apparatus such as printers, display, keypads, sensors and cameras, ROM, a communications sub-system such as a modem, and communications media.

In addition, the above detailed description of embodiments of the invention are not intended to be exhaustive or to limit the invention to the precise form disclosed. For example, while processes or blocks are presented in a given order, alternative embodiments may perform routines having steps, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel, or may be performed at different times.

The teachings of the invention provided herein can be applied to other systems, not necessarily the system described above. The elements and acts of the various embodiments described above can be combined to provide further embodiments.

While some embodiments of the inventions have been described, these embodiments have been presented by way of example only, and are not intended to limit the scope of the disclosure. Indeed, the novel methods and systems described herein may be embodied in a variety of other forms; furthermore, various omissions, substitutions and changes in the form of the methods and systems described herein may be made without departing from the spirit of the disclosure.

### BRIEF DESCRIPTION OF THE DRAWINGS

The invention will now be described in more detail, by way of example, with reference to the accompanying drawings, in which:

FIG. 1a is an image from a camera operating within the thermal image spectrum;

FIG. 1b is an image from a first camera, operating within a first portion of the electromagnetic spectrum, embodying an aspect of the present invention;

FIG. 1c is an image from a second camera, operating within a second portion of the electromagnetic spectrum, embodying an aspect of the present invention;

FIG. 2 is a flow diagram of a user using a touchpoint integrated with a temperature screening system embodying an aspect of the present invention; and

FIG. 3 is a flow diagram of a user interacting with an e-gate integrated with a temperature screening system embodying an aspect of the present invention.

Like features are denoted by like reference numerals.

### DETAILED DESCRIPTION

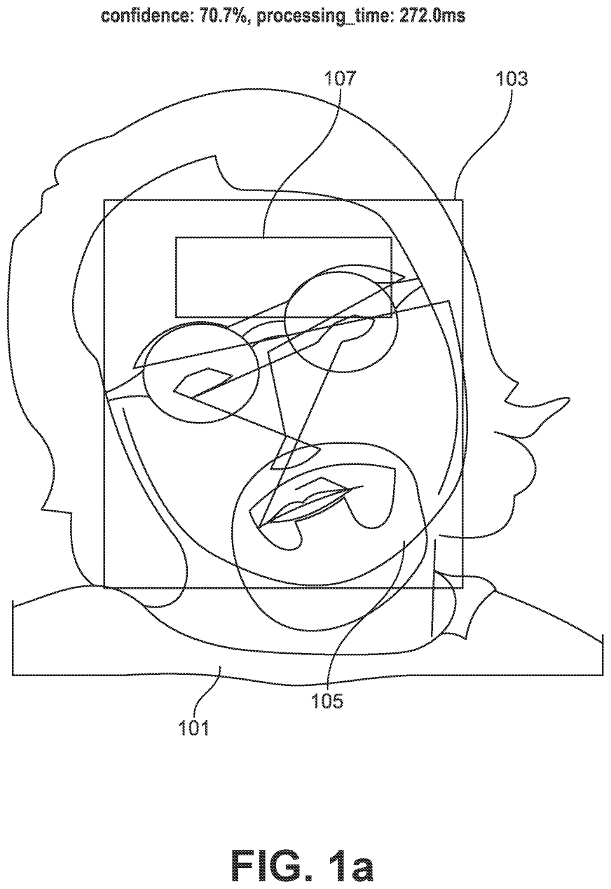

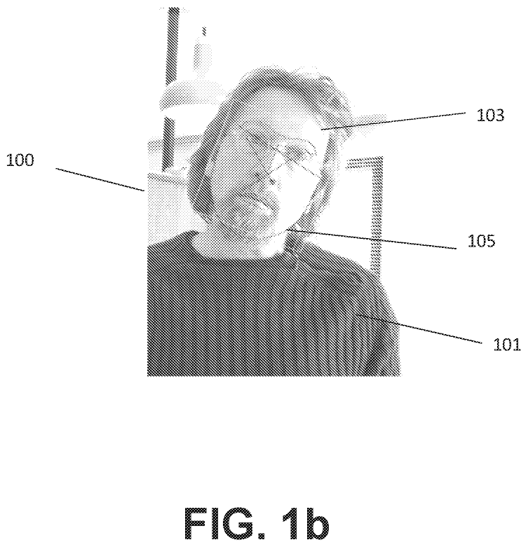

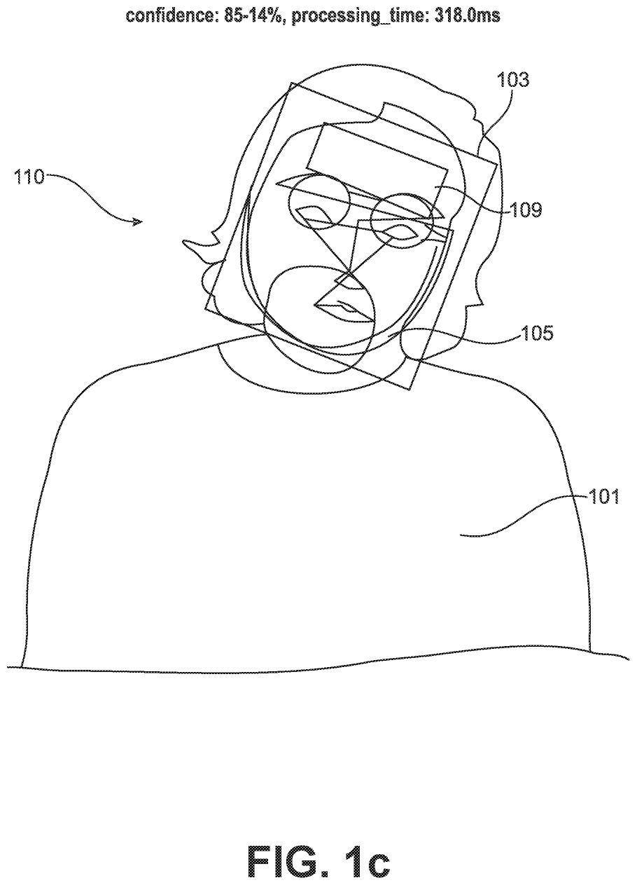

An example system and method for screening a user's temperature will now be described with reference to FIGS. 1a to 3.

FIG. 1a illustrates an image captured with a camera operating within the thermal spectrum. The image captures a user 101, intending to determine the temperature of the user 101 for temperature screening. Overlaid on the image is a facial region 103, facial feature mapping 105, and a temperature region 107 at which a temperature will be measured. The overlays on the image of FIG. 1a demonstrate a sub-optimal mapping, as the thermal image has been taken in isolation and no mapping onto a second image has taken place. Rotation of the user's face has taken place, and therefore the temperature region 107 is not located within a forehead portion of the user's face, but instead overlaps the forehead, the user's eye, and the user's glasses. This would result in a sub-optimal, inaccurate measurement of the user's temperature.

FIGS. 1b and 1c in combination provide more accurate temperature screening. FIGS. 1b and 1c illustrate the use of a temperature screening system according to an embodiment of the present invention. The system comprises a first camera operating within a first portion of the electromagnetic spectrum which in this example is the visible spectrum. The first camera is configured to detect a user's face in a first image 100 (illustrated in FIG. 1b). The system also comprises a second camera operating within a second portion of the electromagnetic spectrum which in this example is the thermal spectrum and is different from the visible spectrum. The second camera is configured to detect the user's face in a second image 110 (illustrated in FIG. 1c). The system also comprises an aligner configured to align the user's face detected in the first image 100 to the user's face detected in the second image 110, and one or more determiners configured to determine: the user's temperature based on the second, thermal spectrum image 110, aligned to the first, visible spectrum image 100; and if the determined user's temperature exceeds a predetermined threshold. The use of this system is described below with respect to FIGS. 1b and 1c.

FIG. 1b is an image captured with a first camera operating within the visible spectrum. The image captures the user 101 in a first image 100. Overlaid on the image is the facial region 103, and facial feature mapping 105.

The user's face is detected in the first image 100 in FIG. 1b and the second image 110 in FIG. 1c. The user's face in the thermal image 110 of FIG. 1c is located using a face location algorithm, which in this example is Thermal Face. The detected user's face from the first image 100 is aligned with the detected user's face in the second image 110 by the aligner using an alignment algorithm. The first camera operating within the visible spectrum provides rotational information indicative of rotation of the user's face about an axis. In this example, the rotation is given as rotation about a horizontal axis located through the middle of the user's face, with a centre of rotation defined at the centre of the user's face. Rotational information is significant as it allows accurate alignment of a temperature portion at which temperature is to be measured, which in this example is a forehead portion 109. The rotational information from the first camera is combined with the location of the face in the second image 110 from the second camera located using the face location algorithm. This allows for an accurate mapping or alignment of the user's face between both images. The forehead portion 109 is determined, and this portion is used for temperature measurement. Preferably embodiments of the perform a rotation correction using the visible image data and perform face location using the infrared image data. Alternatively, a face location algorithm and a rotation correction may be performed on the visible image data. In this case, a linear regression and parallax correction may be applied.

The Parallax Effect

The inventors have appreciated that there may be a parallax effect of the infra-red and visible cameras. For example, if a passenger stands too close to the cameras then there is a discrepancy between what is detected on the visible stream or wavelength which is mapped to the infra-red stream or wavelength. Embodiments of the invention avoid this problem by performing the face location algorithm on the infrared image data.

However, if the visible image data is used, then the parallax effect may be corrected as follows.

Firstly, the number of pixels between the passenger's eyes in the visible wavelength image is determined. The passenger's distance from the cameras may then be estimated using the determined number of pixels, a known typical human eye-to-eye distance of, for example 63 mm, and the camera settings.

The system comprises a plurality of temperature sensors which are calibrated. Once the temperature sensors are calibrated, the temperature is calculated by evaluating the temperature of pixels located in the forehead region. The two pixels with the highest temperature are excluded. Then, the temperature of the five pixels with the next highest temperature are averaged. An offset is then applied to compensate for forehead skin temperature deviation compared to internal body temperature, and the user's temperature is then determined.

Once determined, the measured temperature is compared to a predetermined threshold. The predetermined threshold is determined by averaging temperatures measured on a control set of users or passengers and adding an offset to calculate a threshold. In this example, the threshold indicative of a raised body temperature is any temperature higher than 37.8 degrees Celsius. A user's temperature can therefore be effectively screened using the temperature screening system.

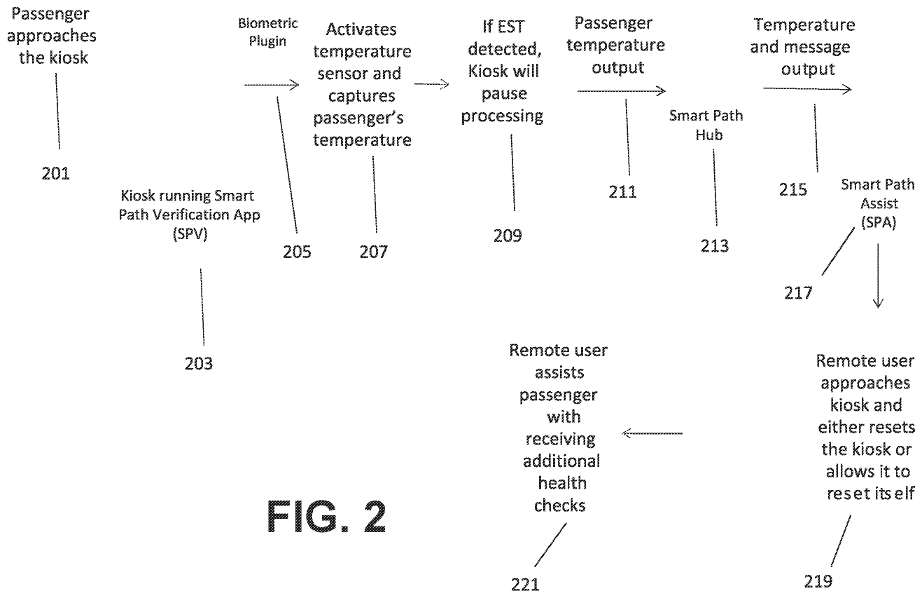

FIG. 2 illustrates a flow diagram of a user's interaction with a smart kiosk located, in this example, in an airport terminal. The smart kiosk is an example of a touchpoint into which the temperature screening system according to the present invention and as described above may be implemented. A similar flow process to that illustrated in FIG. 2 is also applicable to any other touchpoint or point at which the system may be integrated, for example a Facepod, a self-service bag drop, or any other appropriate point in an airport.

At step 201, a user or passenger approaches the kiosk. At step 203, the smart kiosk runs a verification application, which in this example is the Smart Path Verification App (SPV). At step 205, a biometric plugin is used to allow identification of a user, and to allow association of the particular passenger with a subsequently measured temperature. At step 207, the temperature sensor is activated in order to capture the passenger's temperature. The user's temperature is measured by the temperature screening system (which is integrated into the kiosk) in the manner set out above. At step 209, the measured temperature is compared against a predetermined threshold. If the temperature does not exceed the predetermined threshold, the kiosk allows the passenger to proceed further. This may be by any number of means, but in this example, the kiosk issues the passenger with their boarding pass.

However, if the passenger's measured temperature exceeds the predetermined threshold, the kiosk prevents the passenger from proceeding further, for example, by not issuing the passenger with their boarding pass. In either case, the kiosk provides an output comprising the user temperature and an indication of whether the user's temperature exceeds the predetermined threshold at step 211. In this example the measured temperature is output via a wireless local area network. The measured temperature is sent to a database at step 213 to be stored. In this example, a Smart Path Hub comprises the database. The measured temperature is stored together with corresponding user or passenger information, identifying whether or not the passenger has a temperature above the predetermined threshold and therefore considered to have an elevated skin temperature. The database is accessible to a plurality of devices on the network. In this way, where a user approaches any other touchpoint capable of accessing the network, even those without the temperature screening system integrated, the database can be accessed and the user's temperature can be retrieved along with an indication of whether their temperature exceeds the predetermined threshold. In this way, a user with an elevated skin temperature can be prevented from proceeding through the airport, and the passenger's temperature need not be taken at every touchpoint. This may be particularly advantageous where a touchpoint integrated with the temperature screening system is located outside of the airport. A user may have their temperature taken and stored in the database such that, where their temperature is below the predetermined threshold, they may proceed through the airport conveniently and easily without needing their temperature retaken.

Where a measured passenger temperature exceeds the predetermined threshold, the Smart Path Hub provides an output to a remote user on an electronic device at step 215 running an application, which in this example is a Smart Path Assist (SPA) application, at step 217. The remote user oversees or monitors use of a plurality of touchpoints. The remote user is notified that a passenger at one of the touchpoints being monitored has an elevated skin temperature. The remote user then approaches the passenger with a detected elevated skin temperature at step 219. The kiosk is then reset by the remote user or resets itself, ready for use by another passenger. At step 221, the remote user assists the passenger in receiving further health checks to verify whether or not the passenger should be allowed to proceed further through the airport.

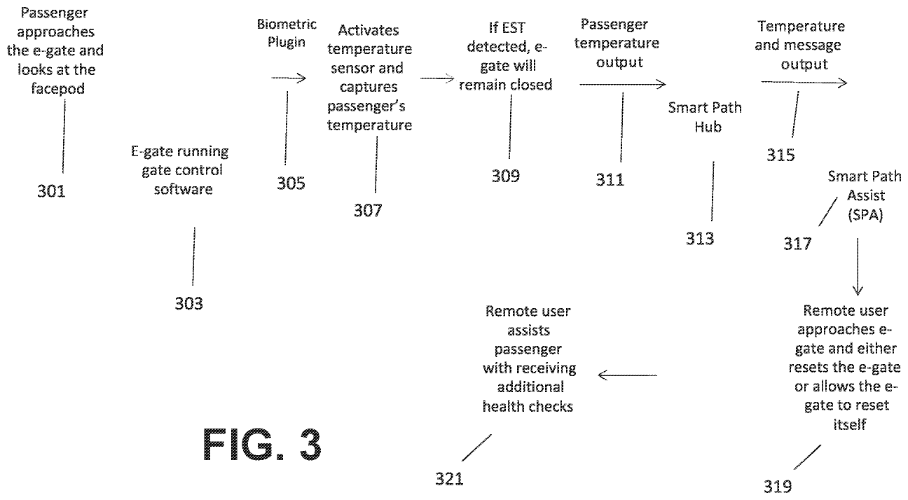

FIG. 3 illustrates a similar flow diagram to that illustrated by FIG. 2, but the temperature screening system is applied in an e-gate located in an airport. The e-gate is an electronic gate which allows or denies access to a passenger. The passenger may need to present a boarding pass or a passport or the like in order to proceed through or past the gate. In this example, the temperature screening system is integrated into the gate and provides a further requirement in order to allow the passenger through the e-gate.

At step 301, the passenger approaches the e-gate, provides their passport, and looks into the Facepod. At step 303, the e-gate runs an application which in this example comprises gate control software. At step 305, a biometric plugin allows identification of the passenger. At step 307, temperature sensors are activated and the passenger's temperature is measured in the manner set out above in relation to FIGS. 1b and 1c.

At step 309, the measured temperature is compared with the predetermined threshold. If the passenger's temperature does not exceed the predetermined threshold, the e-gate allows the user to proceed further through the airport by opening (assuming any other check such as a passport check is passed). However, if the passenger's temperature exceeds the predetermined threshold, the e-gate stays closed and does not allow the passenger to proceed further through the airport.

In either case, the passengers temperature is output at step 311 via a wireless local area network. At step 313, the passengers temperature is stored in a database comprised in the Smart Path Hub. The measured temperature is stored together with corresponding passenger information, associating the passenger with their measured temperature. As explained above, this database can be accessed to retrieve the passenger's temperature via any appropriate electronic device.

At step 315, where the passenger's temperature has exceeded the predetermined threshold, the Smart Path Hub outputs a message or notification to a remote user on an electronic device indicating that a passenger has an elevated skin temperature. Through the electronic device running a Smart Path Assist application at step 317, the remote user can identify the passenger with an elevated skin temperature. At step 319, the remote user approaches the passenger and either resets the e-gate, or the e-gate resets itself, such that the e-gate can be used by another passenger. Lastly, at step 321, the remote user assists the passenger in receiving further health checks to verify whether or not they should proceed further through the airport. The remote user may manually open the e-gate for the user if the additional health checks verify the passengers health.

Embodiments of the present invention have been described. It will be appreciated that variations and modifications may be made to the described embodiments within the scope of the present invention.

Although the above example has been described with reference to a system for use in the aviation industry, it will be clear from the foregoing explanation embodiments of the invention may advantageously be used in any environment where a temperature check needs to be performed, such as a restaurant, cinema, shopping centre, or transportation hub such as rail or bus station.

From the foregoing, it will be appreciated that the system may comprise a computer processor running one or more server processes for communicating with client devices. The server processes comprise computer readable program instructions for carrying out the operations of the present invention. The computer readable program instructions may be or source code or object code written in or in any combination of suitable programming languages including procedural programming languages such as C, object orientated programming languages such as C #, C++, Java, scripting languages, assembly languages, machine code instructions, instruction-set-architecture (ISA) instructions, and state-setting data.

The wired or wireless communication networks described above may be public, private, wired or wireless network. The communications network may include one or more of a local area network (LAN), a wide area network (WAN), the Internet, a mobile telephony communication system, or a satellite communication system. The communications network may comprise any suitable infrastructure, including copper cables, optical cables or fibres, routers, firewalls, switches, gateway computers and edge servers.

The system described above may comprise a Graphical User Interface. Embodiments of the invention may include an on-screen graphical user interface. The user interface may be provided, for example, in the form of a widget embedded in a web site, as an application for a device, or on a dedicated landing web page. Computer readable program instructions for implementing the graphical user interface may be downloaded to the client device from a computer readable storage medium via a network, for example, the Internet, a local area network (LAN), a wide area network (WAN) and/or a wireless network. The instructions may be stored in a computer readable storage medium within the client device.

As will be appreciated by one of skill in the art, the invention described herein may be embodied in whole or in part as a method, a system, or a computer program product including computer readable instructions. Accordingly, the invention may take the form of an entirely hardware embodiment or an embodiment combining software, hardware and any other suitable approach or apparatus.

The computer readable program instructions may be stored on a non-transitory, tangible computer readable medium. The computer readable storage medium may include one or more of an electronic storage device, a magnetic storage device, an optical storage device, an electromagnetic storage device, a semiconductor storage device, a portable an erasable programmable read-only memory (EPROM or Flash memory), a static random access memory (SRAM), a portable compact disc read-only memory (CD-ROM), a digital versatile disk (DVD), a memory stick, a floppy disk.

Exemplary embodiments of the invention may be implemented as a circuit board which may include a CPU, a bus, RAM, flash memory, one or more ports for operation of connected I/O apparatus such as printers, display, keypads, sensors and cameras, ROM, a communications sub-system such as a modem, and communications media.

In addition, the above detailed description of embodiments of the invention are not intended to be exhaustive or to limit the invention to the precise form disclosed. For example, while processes or blocks are presented in a given order, alternative embodiments may perform routines having steps, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel, or may be performed at different times.

The teachings of the invention provided herein can be applied to other systems, not necessarily the system described above. The elements and acts of the various embodiments described above can be combined to provide further embodiments.

While some embodiments of the inventions have been described, these embodiments have been presented by way of example only, and are not intended to limit the scope of the disclosure. Indeed, the novel methods and systems described herein may be embodied in a variety of other forms; furthermore, various omissions, substitutions and changes in the form of the methods and systems described herein may be made without departing from the spirit of the disclosure.

The following numbered clauses are hereby included to give further description of the invention:
1. A method for screening a user temperature, the method comprising:
   detecting a user's face in a first image using a first camera operating within a first portion of the electromagnetic spectrum;
   aligning the detected user's face to a second image of the user's face detected using a second camera operating within a second portion of the electromagnetic spectrum which is different from the first portion of the electromagnetic spectrum;
   determining the user's temperature based on the second image aligned to the first image; and
   determining if the user's temperature exceeds a predetermined threshold.
2. A method according to clause 1 wherein the aligning the detected user's face to the second image of the user's face detected using the second camera comprises using rotational information from the first camera, the rotational information being indicative of rotation of the user's face about an axis.
3. A method according to clause 1 or clause 2 wherein the aligning the detected user's face using the second camera comprises locating the user's face in the second image using a face location algorithm.
4. A method according to any preceding clause wherein the aligning the detected user's face to the second image of the user's face detected using the second camera comprises aligning a forehead portion of the user's face to the second image.
5. A method according to any preceding clause further comprising outputting a user temperature output indicative of whether the user's temperature exceeds the predetermined threshold to a remote user on an electronic device.
6. A method according to clause 5 further comprising, based on the user temperature output, the remote user: allowing the user to proceed further if the user's temperature does not exceed the predetermined threshold; or not allowing the user to proceed further if the user's temperature exceeds the predetermined threshold.
7. A method according to any preceding clause further comprising a self-service device: allowing the user to proceed further if the user's temperature does not exceed the predetermined threshold; or not allowing the user to proceed further if the user's temperature exceeds the predetermined threshold.
8. A method according to any preceding clause wherein the first portion of the electromagnetic spectrum is the visible spectrum, and the second portion of the electromagnetic spectrum is the thermal spectrum.
9. A method according to any preceding clause further comprising storing the user's temperature in a database.
10. A method according to clause 9 wherein the user's temperature is stored in the database together with corresponding user information.
11. A method according to clause 9 or clause 10 further comprising:
   accessing the database;
   retrieving the stored user temperature; and
   allowing the user to proceed further if the user's temperature does not exceed the predetermined threshold, or not allowing the user to proceed further if the user's temperature does exceed the predetermined threshold.
12. A system for screening a user temperature, the system comprising:
   a first camera operating within a first portion of the electromagnetic spectrum and being configured to detect a user's face in a first image;
   a second camera operating within a second portion of the electromagnetic spectrum which is different from the first portion of the electromagnetic spectrum, the second camera being configured to detect the user's face in a second image;
   an aligner configured to align the user's face detected in the first image to the user's face detected in the second image; and
   one or more determiners configured to determine: the user's temperature based on the second image aligned to the first image; and if the determined user's temperature exceeds a predetermined threshold.
13. A system according to clause 12 wherein the aligner is further configured to align the user's face detected in the first image to the user's face detected in the second image using rotational information from the first camera, the rotational information being indicative of rotation of the user's face about an axis.
14. A system according to clause 12 or clause 13 wherein the second camera is configured to detect the user's face in a second image using a face location algorithm.
15. A system according to any of clauses 12 to 14 wherein the aligner is configured to align a forehead portion of the user's face detected in the first image to the user's face detected in the second image.
16. A system according to any of clauses 12 to 15 further comprising an output configured to output a user temperature output indicative of whether the user's temperature exceeds the predetermined threshold to a remote user on an electronic device.
17. A system according to clause 16 wherein the electronic device is configured to receive an input from the remote user: allowing the user to proceed further if the user's temperature does not exceed the predetermined threshold; or not allowing the user to proceed further if the user's temperature exceeds the predetermined threshold.
18. A system according to any of clauses 12 to 17 further comprising a self-service device configured to: allow the user to proceed further if the user's temperature does not exceed the predetermined threshold; or not allow the user to proceed further if the user's temperature exceeds the predetermined threshold.
19. A system according to any of clauses 12 to 18 wherein the first portion of the electromagnetic spectrum is the visible spectrum, and the second portion of the electromagnetic spectrum is the thermal spectrum.
20. A system according to any of clauses 12 to 19 further comprising a database configured to store the user's temperature.
21. A system according to clause 20 wherein the database is configured to store user information corresponding to the user's temperature.
22. A system according to any of clauses 12 to 21 further comprising one or more computing devices configured to:
   access the database;
   retrieve the stored user temperature; and
   allow the user to proceed further if the user's temperature does not exceed the predetermined threshold, or not allow the user to proceed further if the user's temperature does exceed the predetermined threshold.

1. A method for screening a user temperature, the method comprising:
detecting a user's face in a first image captured using a first camera operating within a first portion of the electromagnetic spectrum;
aligning the detected user's face to a second image of the user's face captured using a second camera operating within a second portion of the electromagnetic spectrum which is different from the first portion of the electromagnetic spectrum;
determining the user's temperature based on the second image aligned to the first image; and
determining if the user's temperature exceeds a predetermined threshold.

2. The method according to claim 1, wherein the first portion of the electromagnetic spectrum is the visible spectrum, and the second portion of the electromagnetic spectrum is the thermal spectrum.

3. The method according to claim 1, further comprising aligning of a temperature portion associated or within the second image wherein the second image is associated with thermal image data and wherein the temperature portion defines a portion of the second thermal image at which temperature is to be measured wherein the temperature portion is aligned or rotated by an amount determined from the first image wherein the first image is associated with the visible spectrum image.

4. The method according to claim 1, wherein the second image is associated with thermal image data and wherein the first image is associated with the visible spectrum image and wherein the user's face in the thermal image is located using a thermal face location algorithm.

5. The method according to claim 1, wherein the aligning the detected user's face to the second image of the user's face detected using the second camera comprises using rotational information from the first camera, the rotational information being indicative of rotation of the user's face about an axis.

6. The method according to claim 1, wherein the aligning the detected user's face using the second camera comprises locating the user's face in the second image using a face location algorithm.

7. The method according to claim 1, wherein the aligning the detected user's face to the second image of the user's face detected using the second camera comprises aligning a forehead portion of the user's face associated with the first image to the second image and the method further comprises determining a predetermined number of samples having the highest temperature associated with the data associated with the forehead portion of the user's face associated with the second image and excluding the predetermined number of samples when determining a user's temperature from the data associated with the forehead portion of the user's face.

8. The method according to claim 1, further comprising:
determining a user temperature using data associated with a forehead portion of the user's face associated with the second image; and
performing, via a self-service device, at least one of:
allowing the user to proceed further if the user's temperature does not exceed the predetermined threshold; or
not allowing the user to proceed further if the user's temperature exceeds the predetermined threshold.

9. The method according to claim 1, further comprising:
outputting a user temperature output indicative of whether the user's temperature exceeds the predetermined threshold to a remote user on an electronic device;
based on the user temperature output, the remote user: allowing the user to proceed further if the user's temperature does not exceed the predetermined threshold or not allowing the user to proceed further if the user's temperature exceeds the predetermined threshold; and
store the user's temperature in a database together with corresponding user information.

10. The method according to claim 9, further comprising:
accessing the database;
retrieving the stored user temperature; and
allowing the user to proceed further if the user's temperature does not exceed the predetermined threshold, or not allowing the user to proceed further if the user's temperature does exceed the predetermined threshold.

11. A system for screening a user temperature, the system comprising:
a first camera operating within a first portion of the electromagnetic spectrum wherein the system is configured to detect a user's face in a first image;
a second camera operating within a second portion of the electromagnetic spectrum which is different from the first portion of the electromagnetic spectrum, wherein the system is configured to detect the user's face in a second image;
an aligner configured to align the user's face detected in the first image to the user's face detected in the second image; and
one or more determiners configured to determine: the user's temperature based on the second image aligned to the first image; and if the determined user's temperature exceeds a predetermined threshold.

12. The system according to claim 11, wherein the aligner is further configured to align the user's face detected in the first image to the user's face detected in the second image using rotational information from the first camera, the rotational information being indicative of rotation of the user's face about an axis.

13. The system according to claim 11, wherein the second camera is configured to detect the user's face in a second image using a face location algorithm.

14. The system according to claim 11, wherein the aligner is configured to align a forehead portion of the user's face detected in the first image to the user's face detected in the second image.

15. The system according to claim 11, further comprising an output configured to output a user temperature output indicative of whether the user's temperature exceeds the predetermined threshold to a remote user on an electronic device.

16. The system according to claim 11, wherein at least one of: the electronic device or the self-service device is configured to receive an input from the remote user: allowing the user to proceed further if the user's temperature does not exceed the predetermined threshold; or not allowing the user to proceed further if the user's temperature exceeds the predetermined threshold.

17. The system according to claim 11, wherein the first portion of the electromagnetic spectrum is the visible spectrum, and the second portion of the electromagnetic spectrum is the thermal spectrum.

18. The system according to claim 11, further comprising a database configured to store the user's temperature and store user information corresponding to the user's temperature.

19. The system according to claim 11, further comprising one or more computing devices configured to:
access the database;
retrieve the stored user temperature; and
allow the user to proceed further if the user's temperature does not exceed the predetermined threshold, or not allow the user to proceed further if the user's temperature does exceed the predetermined threshold.

20. The computer readable medium having stored thereon a computer program product which, when executed by one or more computing devices, causes one of the one or more computing devices to:
detect a user's face in a first image captured using a first camera operating within a first portion of the electromagnetic spectrum;
align the detected user's face to a second image of the user's face captured using a second camera operating within a second portion of the electromagnetic spectrum which is different from the first portion of the electromagnetic spectrum;
determine the user's temperature based on the second image aligned to the first image; and
determine if the user's temperature exceeds a predetermined threshold.
